(19) United States
(12) Patent Application Publication
(10) Pub. No.: US 2024/0207281 A1
(43) Pub. Date: Jun. 27, 2024

(54) METHODS OF TREATING B-CELL LYMPHOMA USING COMBINATION THERAPY

(71) Applicants: CELGENE CORPORATION, Summit, NJ (US); BRISTOL-MYERS SQUIBB COMPANY, Princeton, NJ (US)

(72) Inventors: Poliana Alves PATAH, Princeton, NJ (US); Michael POURDEHNAD, San Francisco, CA (US); Xinyu WEI, Summit, NJ (US); Dimitrios ZARDAVAS, Princeton, NJ (US)

(21) Appl. No.: 18/556,566

(22) PCT Filed: Apr. 20, 2022
(86) PCT No.: PCT/US22/25450

§ 371 Date: Oct. 20, 2023

Related U.S. Application Data
(60) Provisional application No. 63/177,639, filed on Apr. 21, 2021.

(51) Int. Cl. A61K 31/5377; A61K 31/475; A61K 31/573; A61K 31/675; A61K 31/704; A61K 39/395; A61P 35/00

(52) U.S. Cl. CPC A61K 31/5377; A61K 31/475; A61K 31/573; A61K 31/675; A61K 31/704; A61K 39/3955; A61P 35/00

(57) ABSTRACT

Provided herein are methods of using 2-(2.6-dioxopiperidin-3-yl)-4-((2-fluoro-4-((3-morpholinoazetidin-1-yl)methyl)benzyl)amino)isoindoline-1, 3-dione, or an enantiomer, a mixture of enantiomers, a tautomer, an isotopolog, or a pharmaceutically acceptable salt thereof, in combination with rituximab, cyclophosphamide, doxorubicin, vincristine, and prednisone or an equivalent thereof for treating, preventing or managing B-cell lymphoma.

### CROSS-REFERENCE TO RELATED APPLICATIONS

This application claims priority to U.S. Provisional Application No. 63/177,639, filed on Apr. 21, 2021, the entirety of which is incorporated herein by reference.

### FIELD

Provided herein are methods of using 2-(2,6-dioxopiperidin-3-yl)-4-((2-fluoro-4-((3-morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with rituximab, cyclophosphamide, doxorubicin, vincristine, and prednisone or an equivalent thereof for treating, preventing or managing B-cell lymphoma.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

All of the current cancer therapy approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells.

With respect to chemotherapy, there are a variety of chemotherapeutic agents available for treatment of cancer. A majority of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove or become refractory to standard chemotherapeutic treatment protocols.

Lymphoma represents a wide spectrum of neoplasms derived from normal lymphoid cells, divided into non-Hodgkin lymphoma (NHL) and Hodgkin lymphoma. World Health Organization (WHO) classification is utilized to define subtypes based on clinical, pathological, phenotypical, and molecular features (Swerdlow et al., Blood 2016, 127(20):2375-90).

Among patients affected by NHL, the majority belong to the aggressive B-cell lymphoma (a-BCL) subtype. Different entities, defined according to the 2016 WHO classification, fall into this category. Most frequent are diffuse large B-cell lymphoma (DLBCL), not otherwise specified (NOS) (including germinal center B-cell [GCB] and activated B-cell [ABC] types); high-grade B-cell lymphoma, with MYC and B-cell lymphoma 2 (BCL2) and/or B-cell lymphoma 6 (BCL6) rearrangements; primary mediastinal (thymic) large B-cell lymphoma (PMBCL); T-cell/histiocyte-rich large B-cell lymphoma; primary cutaneous DLBCL-leg type; intravascular large B-cell lymphoma; anaplastic lymphoma kinase positive (ALK+) large B-cell lymphoma; plasmablastic lymphoma; primary effusion lymphoma (PEL); Epstein Barr virus positive (EBV+) DLBCL, NOS; and rarer subtypes. Grade 3b follicular lymphoma (FL) are included in a-BCL. Diffuse large B-cell lymphoma (DLBCL) and other a-BCLs account for 35% to 40% of NHL cases in North America and Europe.

There remains a significant need for safe and effective methods of treating, preventing and managing aggressive B-cell lymphoma.

Citation or identification of any reference in this section of this application is not to be construed as an admission that the reference is prior art to the present application.

### SUMMARY

Provided herein are methods of using (S)-2-(2,6-dioxopiperidin-3-yl)-4-((2-fluoro-4-((3-morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with a second therapeutic agent, for treating, preventing or managing B-cell lymphoma. The second therapeutic agent is a combination of rituximab, cyclophosphamide, doxorubicin, vincristine, and prednisone or an equivalent thereof. In one embodiment, the second therapeutic agent is R—CHOP.

In certain embodiments, provided herein is a method of treating B-cell lymphoma, comprising administering to a subject in need thereof a therapeutically effective amount of a compound of Formula (I):

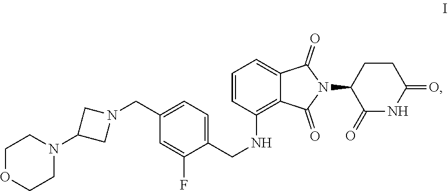 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof, in combination with a second therapeutic agent, wherein the second therapeutic agent is R—CHOP.

In certain embodiments, provided herein is a method of treating B-cell lymphoma, comprising administering to a subject in need thereof a therapeutically effective amount of a hydrochloride salt of a compound of Formula (I), in combination with a second therapeutic agent, wherein the second therapeutic agent is R—CHOP.

In one embodiment, the BCL is aggressive B-cell lymphoma (a-BCL). In one embodiment, the a-BCL is newly diagnosed and/or previously untreated a-BCL.

The present embodiments can be understood more fully by reference to the detailed description and examples, which are intended to exemplify non-limiting embodiments.

### BRIEF DESCRIPTION OF THE DRAWINGS

FIG. 1 shows the dose escalation design of Compound 1 added to R—CHOP-21 regimen for first-line treatment of a-BCL.

FIG. 2 shows dose expansion design of Compound 1 added to R—CHOP-21 regimen for first-line treatment of a-BCL.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of a compound provided herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Others are well-known in the art, see for example, Remington's Pharmaceutical Sciences, 18th eds., Mack Publishing, Easton PA (1990) or Remington: The Science and Practice of Pharmacy, 19th eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments provided herein, including mixtures thereof.

The use of stereomerically pure forms of such compounds, as well as the use of mixtures of those forms, are encompassed by the embodiments provided herein. For example, mixtures comprising equal or unequal amounts of the enantiomers of a particular compound may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag Gmbh & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It is to be understood that the compounds provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

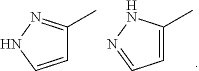

As readily understood by one skilled in the art, a wide variety of functional groups and other structures may exhibit tautomerism and all tautomers of a compound are within the scope of the compound as provided herein.

It should also be noted that a compound provided herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound, whether radioactive or not, are intended to be encompassed within the scope of the compound as provided herein. In some embodiments, provided herein are isotopologues of the compounds, for example, the isotopologues are deuterium, carbon-13 (¹³C), and/or nitrogen-15 (¹⁵N) enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereomerical or isotopic composition, each compound provided herein can be provided in the form of any of the pharmaceutically acceptable salts provided herein. Equally, it is understood that the isotopic composition may vary independently from the stereomerical composition of each compound provided herein. Further, the isotopic composition, while being restricted to those elements present in the respective compound or salt thereof, may otherwise vary independently from the selection of the pharmaceutically acceptable salt of the respective compound.

It should be noted that if there is a discrepancy between a depicted structure and a name for that structure, the depicted structure is to be accorded more weight.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with a compound means an amount capable of treating, preventing, or managing a disorder, disease or condition, or symptoms thereof.

As used herein and unless otherwise indicated, the terms “co-administration” and “in combination with” include the administration of one or more therapeutic agents (for example, a compound provided herein and another anti-BCL agent, cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms.

As used herein and unless otherwise specified, “a therapeutic agent” provided herein is not limited to a single therapeutic agent, and it can be, in certain embodiments, a combination of one or more different therapeutic agents. The one or more therapeutic agents can be administered in combination with each other as described herein. As used herein and unless otherwise specified, “a therapeutic agent” can be used interchangeably with “a therapeutic therapy”, and is not limited to a therapeutic substance. For example, a therapeutic agent can be a cancer treatment such as radiation therapy or CAR-T therapy.

An “cycling therapy” refers to a regimen or therapy that includes an administration period as described herein and optionally a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

As used herein and unless otherwise indicated, the term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

In the context of a cancer, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates is computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response ≥partial response (PR). In one embodiment, duration of response (DoR) is the time from achieving a response until relapse or disease progression. In one embodiment, DoR is the time from achieving a response ≥partial response (PR) until relapse or disease progression. In one embodiment, DoR is the time from the first documentation of a response until to the first documentation of progressive disease or death. In one embodiment, DoR is the time from the first documentation of a response ≥partial response (PR) until to the first documentation of progressive disease or death. In one embodiment, time to response (TTR) means the time from the first dose of compound to the first documentation of a response. In one embodiment, TTR means the time from the first dose of compound to the first documentation of a response ≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of NHL may be assessed by the International Workshop Criteria for Malignant Lymphoma (see Cheson et al., J. Clin. Oncol.. 2014, 32(27):3059-3068) and the Deauville Criteria for fluorodeoxyglucose-positron emission tomography (FDG-PET) scan interpretation (Itti et al., Eur. J. Nucl. Med. Mol. Imaging, 2013, 40(9): 1312-20; Meignan et al., Leuk Lymphoma, 2014, 55(1):31-37) (“Lugano criteria”), using the response and end point definition shown in Tables 1-3.

TABLE 1
Criteria for Involvement of Site.

| Tissue Site | Clinical | FDG Avidity | Test | Positive Finding |
|---|---|---|---|---|
| Lymph nodes | Palpable | FDG-avid histologies | PET/CT | Increase FDG uptake |
| | | Nonavid disease | CT | Unexplained node |
| | | | | enlargement |
| Spleen | Palpable | FDG-avid histologies | PET/CT | Diffuse uptake, solitary |
| | | | | mass, miliary lesions, |
| | | | | nodules |
| | | Nonavid disease | CT | >13 cm in vertical length, |
| | | | | mass, nodules |
| Liver | Palpable | FDG-avid histologies | PET/CT | Diffuse uptake, mass |
| | | Nonavid disease | CT | Nodules |
| CNS | Signs, | N/A | CT | Mass lesion(s) |
| | symptoms | | MRI | Leptomeningeal |
| | | | | infiltration, mass lesions |
| | | | CSF | Cytology, flow cytometry |
| | | | assessment | |
| Other (eg, skin, | Site | N/A | PET/CT^{a}, | Lymphoma involvement |
| lung, GI tract, | dependent | | biopsy | |
| bone, bone | | | | |
| marrow) | | | | |

CNS = central nervous system; CSF = cerebrospinal fluid; CT = computed tomography; FDG = fluorodeoxyglucose; GI = gastrointestinal; MRI = magnetic resonance imaging; PET = positron emission tomography; N/A = not applicable.
^{a}PET/CT is adequate for determination of bone marrow involvement and can considered highly suggestive for involvement of other extralymphatic sites. Biopsy confirmation of those sites can be considered if necessary.

TABLE 2
Lugano Response Criteria for Non-Hodgkin Lymphoma.

| Response | Site | PET/CT (metabolic response) | CT (Radiologic response) |
|---|---|---|---|
| Complete | Lymph nodes | Score 1, 2, 3 with or without | All of the following: |
| response | and | residual mass on 5-PS (Table 3) | Target nodes/nodal masses must regress |
| | extralymphatic | | to ≤1.5 cm in LDi |
| | sites | | No extralymphatic sites of disease |
| | Non-measured | N/A | Absent |
| | lesion | | |
| | Organ | N/A | Regress to normal |
| | enlargement | | |
| | New Lesions | None | None |
| | Bone Marrow | No evidence of FDG-avid disease | Normal by morphology; if inderterminate, |
| | | in marrow | IHC negative^{a} |
| Partial | Lymph nodes | Score 4 or 5 on 5-PS with reduced | All of the following: ≥50% |
| Response | and | uptake compared with baseline and | decrease in SPD of up to 6 target |
| | extralymphatic | residual mass(es) of any size | measureable nodes and extranodal sites |
| | sites | At interim these findings suggest | When a lesion is too small to measure on |
| | | responding disease | CT, assign 5 mm × 5 mm as the default |
| | | At end of treatment these findings | value |
| | | may indicate residual disease | When no longer visible, 0 mm × 0 mm |
| | | | For a node >5 mm × 5 mm, but smaller |
| | | | than normal, use actual measurement for |
| | | | calculation |
| | Non-measured | N/A | Absent/normal, regressed, but no increase |
| | lesion | | |
| | Organ | N/A | Spleen must have regressed by >50% in |
| | enlargement | | length beyond normal |
| | New Lesions | None | None |
| | Bone Marrow | Residual uptake higher than uptake | N/A |
| | | in normal marrow but reduced | |
| | | compared with baseline. If | |
| | | persistent focal changes in the | |
| | | marrow in the context of nodal | |
| | | response, consider MRI or biopsy | |
| | | or interval scan | |
| Stable | Target | Score 4 or 5 on 5-PS with no | <50% decrease from baseline of up to 6 |
| Disease | nodes/nodal | significant change in FDG uptake | dominant, measureable nodes and |
| | masses, | from baseline | extranodal sites |
| | extranodal | | No criteria for progressive disease are met |
| | lesions | | |
| | Non-measured | N/A | No increase consistent with progression |
| | lesion | | |
| | Organ | N/A | No increase consistent with progression |
| | enlargement | | |
| | New Lesions | None | None |
| | Bone Marrow | No change from baseline | N/A |
| Progressive | Lymph nodes | Score 4 or 5 on 5-PS with an | At least one of the following: |
| Disease | and | increase in intensity of uptake | PPD progression: |
| | extralymphatic | compared with baseline | An individual node/lesion must be |
| | sites | and/or | abnormal with: |
| | | New FDG-avid foci consistent with | LDi >1.5 cm and |
| | | lymphoma | Increase by ≥50% from PPD nadir and |
| | | | An increase in LDi or SDi from nadir |
| | | | 0.5 cm for lesions ≤2 cm |
| | | | 1.0 cm for lesions >2 cm |
| | | | In the setting of splenomegaly, splenic |
| | | | length must increase by >50% of the extent |
| | | | of its prior increase above baseline (eg, a |
| | | | 15 cm spleen must increase to >16 cm). If |
| | | | no splenomegaly, must increase by at least |
| | | | 2 cm from baseline must increase by at |
| | | | least 2 cm from baseline |
| | | | New or recurrent splenomegaly |
| | Non-measured | None | New or clear progression of preexisting |
| | lesion | | nonmeasured lesions |
| | New Lesions | New FDG-avid foci consistent with | Regrowth of previously resolved lesions |
| | | lymphoma rather than another | A new node >1.5 cm in any axis |
| | | etiology (eg, infection, | A new extranodal site >1.0 cm in any axis; |
| | | inflammation). If uncertain | if <1.0 cm in any axis, its presence must be |
| | | etiology, consider biopsy or | unequivocal and must be attributable to |
| | | interval scan | lymphoma |
| | | | Assessable disease of any size |
| | | | unequivocally attributable to lymphoma |
| | Bone Marrow | New of recurrent FDG-avid foci | New or recurrent involvement |

CMR = complete metabolic response; LDi = longest transverse diameter of a lesion; PPD = cross product of the LDi and perpendicular diameter; SDi = shortest axis perpendicular to the LDi; SPD = sum of the product of the perpendicular diameters for multiple lesions; N/A = not applicable.
^{a}Required for CR if bone marrow involvement at baseline
^{b} In Waldeyer's ring or extranodal sites with high physiologic uptake or with activation within spleen or marrow; (eg with chemotherapy or myeloid colony stimulating factors), uptake may be greater than normal mediastinum and/or liver. In this circumstance, CMR may be inferred if uptake at sites of initial involvement is no greater than surrounding normal tissue.
^{c} FDG-avid lymphomas should have response assessed by PET-CT. Some diseases can typically be followed with CT alone (ie, marginal zone lymphoma).
^{d} PET should be done with contrast-enhanced diagnostic CT and can be done simultaneously or at separate procedures.

TABLE 3
PET Five Point Scale (5-PS).

| | |
|---|---|
| 1 | No uptake above background |
| 2 | Uptake ≤ mediastinum |
| 3 | Uptake > mediastinum but ≤ liver |
| 4 | Uptake moderately > liver |
| 5 | Uptake markedly higher than liver and/or new lesions |
| X | New areas of uptake unlikely to be related to lymphoma |

^{a} The Deauville five-point scale (5PS) is an internationally recommended scale for clinical routine and clinical trials using FDG-PET/CT in the initial staging and assessment of treatment response in Hodgkin lymphoma (HL) and certain types of non-Hodgkin lymphomas (NHL).

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography) scan, MRI (magnetic resonance imaging) Brain/Spine, CSF (cerebrospinal fluid), ophthalmologic exams, vitreal fluid sampling, retinal photograph, bone marrow evaluation and other commonly accepted evaluation modalities.

The term “supportive care agent” refers to any substance that treats, prevents or manages an adverse effect from treatment with another therapeutic agent.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In one embodiment, the terms “about” and “approximately,” when used in this context, contemplate a dose, amount, or weight percent within 30%, within 20%, within 15%, within 10%, or within 5%, of the specified dose, amount, or weight percent.

### Compounds

In one embodiment, the compound used in the methods provided herein is (S)-2-(2,6-dioxopiperidin-3-yl)-4-((2-fluoro-4-((3-morpholinoazetidin-1-yl)methyl)benzyl)amino)isoindoline-1,3-dione of the following formula:

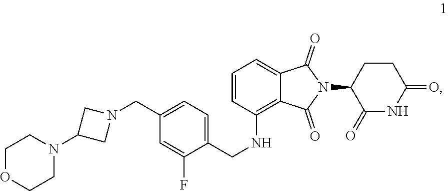 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof. (S)-2-(2,6-dioxopiperidin-3-yl)-4-((2-fluoro-4-((3-morpholinoazetidin-1-yl)methyl)benzyl)amino)isoindoline-1,3-dione is also referred herein as “Compound 1.”

In one embodiment, the compound used in the methods provided herein is (R)-2-(2,6-dioxopiperidin-3-yl)-4-((2-fluoro-4-((3-morpholinoazetidin-1-yl)methyl)benzyl)amino)isoindoline-1,3-dione of the following formula (referred herein as “Compound 2”):

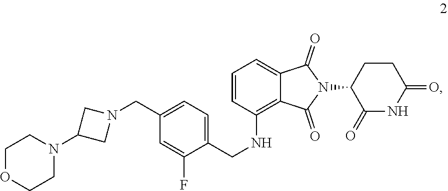 [SMILES: O=C1CC[C@@H](N2C(=O)c3cccc(NCc4ccc(CN5CC(N6CCOCC6)C5)cc4F)c3C2=O)C(=O)N1]

or tautomer, isotopolog, or pharmaceutically acceptable salt thereof.

In one embodiment, the compound used in the methods provided herein is 2-(2,6-dioxopiperidin-3-yl)-4-((2-fluoro-4-((3-morpholinoazetidin-1-yl)methyl)benzyl)amino)isoindoline-1,3-dione of the following formula (referred herein as “Compound 3”):

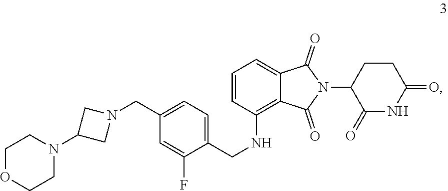 [SMILES: O=C1CCC(N2C(=O)c3cccc(NCc4ccc(CN5CC(N6CCOCC6)C5)cc4F)c3C2=O)C(=O)N1]

or tautomer, isotopolog, or pharmaceutically acceptable salt thereof.

In one embodiment, Compound 1 is used in the methods provided herein. In one embodiment, a tautomer of Compound 1 is used in the methods provided herein. In one embodiment, an isotopolog of Compound 1 is used in the methods provided herein. In one embodiment, a pharmaceutically acceptable salt of Compound 1 is used in the methods provided herein. In one embodiment, a hydrochloride salt of Compound 1 is used in the methods provided herein. In one embodiment, a mono-hydrochloride salt of Compound 1 is used in the methods provided herein. Certain salts and polymorphic forms of Compound 1 are described in U.S. patent application Ser. No. 17/075,359, the entirety of which is incorporated herein by reference.

In one embodiment, Compound 2 is used in the methods provided herein. In one embodiment, a tautomer of Compound 2 is used in the methods provided herein. In one embodiment, an isotopolog of Compound 2 is used in the methods provided herein. In one embodiment, a pharmaceutically acceptable salt of Compound 2 is used in the methods provided herein. In one embodiment, a hydrochloride salt of Compound 2 is used in the methods provided herein.

In one embodiment, Compound 3 is used in the methods provided herein. In one embodiment, an enantiomer of Compound 3 is used in the methods provided herein. In one embodiment, a mixture of enantiomers of Compound 3 is used in the methods provided herein. In one embodiment, a tautomer of Compound 3 is used in the methods provided herein. In one embodiment, an isotopolog of Compound 3 is used in the methods provided herein. In one embodiment, a pharmaceutically acceptable salt of Compound 3 is used in the methods provided herein. In one embodiment, a hydrochloride salt of Compound 3 is used in the methods provided herein.

The synthesis and certain use of the compounds provided herein are described in U.S. Patent Publication Nos. 2019/0322647 A1 and 2020/0325129 A1, and U.S. patent application Ser. Nos. 17/075,496, 17/075,523, and 17/075,125, the entirety of each of which is incorporated herein by reference.

### Methods of Treatment and Prevention

In one embodiment, provided herein are methods of using (S)-2-(2,6-dioxopiperidin-3-yl)-4-((2-fluoro-4-((3-morpholinoazetidin-1-yl)methyl)benzyl)amino)isoindoline-1,3-dione, or an enantiomer, a mixture of enantiomers, a tautomer, an isotopolog, or a pharmaceutically acceptable salt thereof, in combination with a second therapeutic agent, for treating, preventing or managing BCL. In one embodiment, the second therapeutic agent is a combination of rituximab, cyclophosphamide, doxorubicin, vincristine, and prednisone or an equivalent thereof.

In one embodiment, provided herein is a method of treating BCL, comprising administering to a subject in need thereof a therapeutically effective amount of a compound of Formula (I):

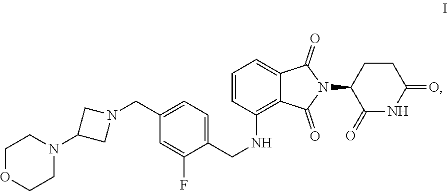 [SMILES: O=C1CC[C@H](N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof (e.g., a hydrochloride salt), in combination with a second therapeutic agent, wherein the second therapeutic agent is a combination of rituximab, cyclophosphamide, doxorubicin, vincristine, and prednisone or an equivalent thereof. Unless otherwise specified, “a compound of Formula (I)” and “Compound 1” are used interchangeably herein.

In one embodiment, provided herein is a method of preventing BCL, which comprises administering to a subject in need thereof a therapeutically effective amount of Compound 1, or an enantiomer, mixture of enantiomers, tautomer, isotopolog, or pharmaceutically acceptable salt thereof (e.g., a hydrochloride salt), in combination with a second therapeutic agent, wherein the second therapeutic agent is a combination of rituximab, cyclophosphamide, doxorubicin, vincristine, and prednisone or an equivalent thereof.

In one embodiment, provided herein is a method of managing BCL, which comprises administering to a subject in need thereof a therapeutically effective amount of Compound 1, or an enantiomer, mixture of enantiomers, tautomer, isotopolog, or pharmaceutically acceptable salt thereof (e.g., a hydrochloride salt), in combination with a second therapeutic agent, wherein the second therapeutic agent is a combination of rituximab, cyclophosphamide, doxorubicin, vincristine, and prednisone or an equivalent thereof.

In certain embodiments, the BCL is aggressive B-cell lymphoma (a-BCL). In one embodiment, the aggressive B-cell lymphoma is as determined according to 2016 WHO classification (Swerdlow et al., Blood 2016, 127(20):2375-90), the entirety of which is incorporated herein by reference.

In some embodiments, the BCL is primary mediastinal (thymic) large B-cell lymphoma (PMBCL).

In some embodiments, the BCL is primary cutaneous DLBCL. In some embodiments, the BCL is primary cutaneous DLBCL-leg type.

In some embodiments, the BCL is anaplastic lymphoma kinase positive (ALK+) large B-cell lymphoma.

In some embodiments, the BCL is follicular lymphoma (FL). In some embodiments, the BCL is Grade 3b follicular lymphoma (FL).

In some embodiments, the BCL is diffuse large B-cell lymphoma (DLBCL). In one embodiment, the DLBCL is DLBCL not otherwise specified (NOS). In one embodiment, the DLBCL is germinal center B-cell (GCB) type. In one embodiment, the DLBCL is activated B-cell (ABC) type.

In some embodiments, the BCL is high-grade B-cell lymphoma. In one embodiment, the high-grade B-cell lymphoma has MYC rearrangement. In one embodiment, the high-grade B-cell lymphoma has BCL2 rearrangement. In one embodiment, the high-grade B-cell lymphoma has BCL6 rearrangement. In one embodiment, the high-grade B-cell lymphoma has MYC and BCL2 and/or BCL6 rearrangements. As used herein and unless otherwise specified, high-grade B-cell lymphoma having “MYC and BCL2 and/or BCL6 rearrangements” means the high-grade B-cell lymphoma has MYC rearrangement and either or both of BCL2 and BCL6 rearrangements. In one embodiment, the high-grade B-cell lymphoma has MYC and BCL2 rearrangements. In one embodiment, the high-grade B-cell lymphoma has MYC and BCL6 rearrangements. In one embodiment, the high-grade B-cell lymphoma has MYC, BCL2, and BCL6 rearrangements.

In some embodiments, the BCL is Epstein Barr virus positive (EBV+) DLBCL. In one embodiment, the EBV+DLBCL is EBV+DLBCL not otherwise specified (NOS).

In one embodiment, the BCL is T-cell/histiocyte-rich large B-cell lymphoma (THRLBCL). In one embodiment, the BCL is intravascular large B-cell lymphoma. In one embodiment, the BCL is plasmablastic lymphoma. In one embodiment, the BCL is primary effusion lymphoma (PEL).

The International Prognostic Index (IPI) score an important prognostic tool. Five clinical characteristics (age, lactate dehydrogenase [LDH], number of extra nodal sites, Ann Arbor stage, and Eastern Cooperative Oncology Group [ECOG] performance status) are used to stratify patients into the following 4 risk categories: low risk (0 to 1 risk factor), low-intermediate risk (2 risk factors), high intermediate risk (3 risk factors) and high risk (4 to 5 risk factors). Other prognostic factors include factors associated with phenotypic or molecular features of lymphoma cells such as cell of origin, double-hit and double-expression, and tumor microenvironment.

In one embodiment, the BCL is poor risk aggressive B-cell lymphoma. In one embodiment, the BCL is high risk aggressive B-cell lymphoma. In one embodiment, the BCL has International Prognostic Index (IPI) score of 3 to 5. In one embodiment, the BCL has IPI score of 3. In one embodiment, the BCL has IPI score of 4. In one embodiment, the BCL has IPI score of 5.

In one embodiment, the BCL is previously untreated. In one embodiment, the BCL is previously untreated aggressive B-cell lymphoma. In one embodiment, the BCL is previously untreated DLBCL, NOS (including GCB and ABC types). In one embodiment, the BCL is previously untreated high-grade B-cell lymphoma with MYC and BCL2 and/or BCL6 rearrangements. In one embodiment, the BCL is previously untreated PMBCL. In one embodiment, the BCL is previously untreated primary cutaneous DLBCL-leg type. In one embodiment, the BCL is previously untreated ALK+large B-cell lymphoma. In one embodiment, the BCL is previously untreated EBV+DLBCL, NOS. In one embodiment, the BCL is previously untreated grade 3b follicular lymphoma.

In one embodiment, the BCL is newly diagnosed. In one embodiment, the BCL is newly diagnosed aggressive B-cell lymphoma. In one embodiment, the BCL is newly diagnosed DLBCL, NOS (including GCB and ABC types). In one embodiment, the BCL is newly diagnosed high-grade B-cell lymphoma with MYC and BCL2 and/or BCL6 rearrangements. In one embodiment, the BCL is newly diagnosed PMBCL. In one embodiment, the BCL is newly diagnosed primary cutaneous DLBCL-leg type. In one embodiment, the BCL is newly diagnosed ALK+large B-cell lymphoma. In one embodiment, the BCL is newly diagnosed EBV+DLBCL, NOS. In one embodiment, the BCL is newly diagnosed grade 3b follicular lymphoma.

In one embodiment, a compound provided herein (e.g., Compound 1, or a pharmaceutically acceptable salt there of (e.g., a hydrochloride salt)) is administered in combination with a second therapeutic agent provided herein as a first line treatment of the BCL.

In one embodiment, the second therapeutic agent used in the methods provided herein is a combination of rituximab, cyclophosphamide, doxorubicin, vincristine, and prednisone or an equivalent thereof.

In certain embodiments, the second therapeutic agent is R—CHOP. As used herein and unless otherwise specified, R—CHOP therapy refers to chemotherapy with a course of rituximab, cyclophosphamide, doxorubicin, vincristine, and prednisone (or an equivalent such as prednisolone). In certain embodiments, R—CHOP therapy is given as a course of several cycles of treatment over a few months. In certain embodiments, each cycle of R—CHOP is 21 days (three weeks), wherein rituximab, cyclophosphamide, doxorubicin, and vincristine are administered on the first day of the 21-day cycle, and a five-day course of prednisone (or prednisolone) is also started on Days 1-5 of the 21-day cycle.

In one embodiment, prednisone is administered in the methods provided herein. In one embodiment, an equivalent of prednisone is administered in place of prednisone. In one embodiment, prednisolone is administered. In one embodiment, a corticosteroid equivalent of prednisone is administered. In one embodiment, the corticosteroid equivalent of prednisone is administered is administered intravenously (e.g., on day 1 of the cycle for convenience).

In certain embodiments, the second therapeutic agent is a combination of rituximab, cyclophosphamide, doxorubicin, vincristine, and prednisone.

In certain embodiments, the second therapeutic agent is a combination of rituximab, cyclophosphamide, doxorubicin, vincristine, and prednisolone.

In certain embodiments, the doxorubicin is not liposomal doxorubicin.

Rituximab is an anti-CD20 monoclonal antibody. In one embodiment, rituximab is administered in an amount according to the physician's decision. In one embodiment, rituximab is administered according to the locally approved label or pharmacy manual for preparation, administration, and storage information. In one embodiment, rituximab is administered according to the label of RITUXAN®. In one embodiment, rituximab is administered at a dose of about 375 mg/m² per day. In one embodiment, rituximab is administered at a dose of about 1400 mg per day. In one embodiment, rituximab is administered on day 1 of a 21-day cycle. In one embodiment, rituximab is administered intravenously. In one embodiment, rituximab is administered via intravenous injection. In one embodiment, rituximab is administered via intravenous infusion. In one embodiment, rituximab is administered subcutaneously. In one embodiment, rituximab is administered via subcutaneous infusion.

In one embodiment, rituximab is administered intravenously at a dose of about 375 mg/m² per day on day 1 of a 21-day cycle. In one embodiment, rituximab is administered subcutaneously at a dose of about 1400 mg per day on day 1 of a 21-day cycle.

In one embodiment, cyclophosphamide is administered in an amount according to the physician's decision. In one embodiment, cyclophosphamide is administered according to the locally approved label or pharmacy manual for preparation, administration, and storage information. In one embodiment, cyclophosphamide is administered according to the label of CYTOXAN®. In one embodiment, cyclophosphamide is administered according to the label of NEOSAR®. In one embodiment, cyclophosphamide is administered at a dose of about 750 mg/m² per day. In one embodiment, cyclophosphamide is administered on day 1 of a 21-day cycle. In one embodiment, cyclophosphamide is administered intravenously. In one embodiment, cyclophosphamide is administered via intravenous injection. In one embodiment, cyclophosphamide is administered via intravenous infusion.

In one embodiment, cyclophosphamide is administered intravenously at a dose of about 750 mg/m² per day on day 1 of a 21-day cycle.

In one embodiment, doxorubicin is administered in an amount according to the physician's decision. In one embodiment, doxorubicin is administered according to the locally approved label or pharmacy manual for preparation, administration, and storage information. In one embodiment, doxorubicin is administered according to the label of ADRIAMYCIN®. In one embodiment, doxorubicin is administered according to the label of RUBEX®. In one embodiment, doxorubicin is administered at a dose of about 50 mg/m² per day. In one embodiment, doxorubicin is administered on day 1 of a 21-day cycle. In one embodiment, doxorubicin is administered intravenously. In one embodiment, doxorubicin is administered via intravenous injection. In one embodiment, doxorubicin is administered via intravenous infusion.

In one embodiment, doxorubicin is administered intravenously at a dose of about 50 mg/m² per day on day 1 of a 21-day cycle.

In one embodiment, vincristine is administered in an amount according to the physician's decision. In one embodiment, vincristine is administered according to the locally approved label or pharmacy manual for preparation, administration, and storage information. In one embodiment, vincristine is administered according to the label of ONCOVIN®. In one embodiment, vincristine is administered at a dose of about 1.4 mg/m² per day. In one embodiment, vincristine is administered with a maximum amount of 2.0 mg per day. In one embodiment, vincristine is administered on day 1 of a 21-day cycle. In one embodiment, vincristine is administered intravenously. In one embodiment, vincristine is administered via intravenous injection. In one embodiment, vincristine is administered via intravenous infusion.

In one embodiment, vincristine is administered intravenously at a dose of about 1.4 mg/m² per day on day 1 of a 21-day cycle with a maximum amount of 2.0 mg per day.

In one embodiment, prednisone or an equivalent thereof is administered in an amount according to the physician's decision. In one embodiment, prednisone or an equivalent thereof is administered according to the locally approved label or pharmacy manual for preparation, administration, and storage information. In one embodiment, prednisone or an equivalent thereof is administered according to the label of DELTASONE®. In one embodiment, prednisone or an equivalent thereof is administered at a dose of about 100 mg per day. In one embodiment, prednisone or an equivalent thereof is administered on days 1 to 5 of the 21-day cycle. In one embodiment, prednisone or an equivalent thereof is administered intravenously. In one embodiment, prednisone or an equivalent thereof is administered via intravenous injection. In one embodiment, prednisone or an equivalent thereof is administered via intravenous infusion. In one embodiment, prednisone or an equivalent thereof is administered orally. In one embodiment, prednisone or an equivalent thereof is administered orally with food. In one embodiment, prednisone or an equivalent thereof is administered orally without food.

In one embodiment, prednisone is administered orally at a dose of about 100 mg per day on days 1 to 5 of a 21-day cycle. In one embodiment, prednisone is administered at a dose of about 100 mg per day intravenously on day 1 of a 21-day cycle and orally on days 2 to 5 of the 21-day cycle. In one embodiment, prednisolone is administered orally at a dose of about 100 mg per day on days 1 to 5 of a 21-day cycle. In one embodiment, prednisolone is administered at a dose of about 100 mg per day intravenously on day 1 of a 21-day cycle and orally on days 2 to 5 of the 21-day cycle.

In one embodiment, rituximab, cyclophosphamide, doxorubicin, and vincristine are administered on day 1 of a 21-day cycle, and prednisone or an equivalent thereof is administered on days 1 to 5 of the 21-day cycle.

In one embodiment, rituximab is administered intravenously or subcutaneously; cyclophosphamide, doxorubicin, and vincristine are administered intravenously; and prednisone or an equivalent thereof is administered orally.

In one embodiment, rituximab is administered intravenously at a dose of about 375 mg/m², or subcutaneously at a dose of about 1400 mg, on day 1 of a 21-day cycle; cyclophosphamide is administered intravenously at a dose of about 750 mg/m² on day 1 of the 21-day cycle; doxorubicin is administered intravenously at a dose of about 50 mg/m² on day 1 of the 21-day cycle; vincristine is administered intravenously at a dose of about 1.4 mg/m² on day 1 of the 21-day cycle; and prednisone or an equivalent thereof is administered orally at a dose of about 100 mg on days 1 to 5 of the 21-day cycle.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) to the administration of a second therapeutic agent provided herein to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered concomitantly with the administration of a second therapy provided herein to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent provided herein to the subject.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of from about 0.2 mg to about 0.6 mg per day. In one embodiment, the compound is administered at a dose of from about 0.2 mg to about 0.4 mg per day. In one embodiment, the compound is administered at a dose of from about 0.4 mg to about 0.6 mg per day.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of from about 0.2 mg to about 0.6 mg once daily. In one embodiment, the compound is administered at a dose of from about 0.2 mg to about 0.4 mg once daily. In one embodiment, the compound is administered at a dose of from about 0.4 mg to about 0.6 mg once daily.

In certain embodiments, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of about 0.2 mg, about 0.3 mg, about 0.4 mg, about 0.5 mg, or about 0.6 mg per day. In certain embodiments, the compound is administered at a dose of about 0.2 mg per day. In certain embodiments, the compound is administered at a dose of about 0.3 mg per day. In certain embodiments, the compound is administered at a dose of about 0.4 mg per day. In certain embodiments, the compound is administered at a dose of about 0.5 mg per day. In certain embodiments, the compound is administered at a dose of about 0.6 mg per day.

In certain embodiments, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of about 0.2 mg, about 0.3 mg, about 0.4 mg, about 0.5 mg, or about 0.6 mg once daily. In certain embodiments, the compound is administered at a dose of about 0.2 mg once daily. In certain embodiments, the compound is administered at a dose of about 0.3 mg once daily. In certain embodiments, the compound is administered at a dose of about 0.4 mg once daily. In certain embodiments, the compound is administered at a dose of about 0.5 mg once daily. In certain embodiments, the compound is administered at a dose of about 0.6 mg once daily.

In one embodiment, a compound of Formula (I), or a pharmaceutically acceptable salt thereof, is administered. In one embodiment, a hydrochloride salt of a compound of Formula (I) is administered.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered orally. In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered after an overnight fast lasting for at least 6 hours (e.g., in the morning with approximately 8 oz or 240 ml of water). In one embodiment, the subject refrains from food or other medication intake for at least 2 hours after the compound is administered.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered once daily for 7 days, followed by 14 days of rest. In on embodiment, the compound is administered once daily for 10 days, followed by 11 days of rest.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered on days 1 to 7 of a 21-day cycle. In one embodiment, the compound is administered on days 1 to 10 of a 21-day cycle. In one embodiment, the administration period of the compound is followed by rest of the compound on the remaining days of the 21-day cycle.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of from about 0.2 mg to about 0.6 mg once daily on days 1 to 7 of a 21-day cycle. In one embodiment, the compound is administered at a dose of about 0.2 mg once daily on days 1 to 7 of a 21-day cycle. In one embodiment, the compound is administered at a dose of about 0.3 mg once daily on days 1 to 7 of a 21-day cycle. In one embodiment, the compound is administered at a dose of about 0.4 mg once daily on days 1 to 7 of a 21-day cycle. In one embodiment, the compound is administered at a dose of about 0.5 mg once daily on days 1 to 7 of a 21-day cycle. In one embodiment, the compound is administered at a dose of about 0.6 mg once daily on days 1 to 7 of a 21-day cycle. In one embodiment, the administration period of the compound is followed by rest of the compound on days 8 to 21 of the 21-day cycle.

In one embodiment, a compound described herein, e.g., Compound 1, or an enantiomer, mixture of enantiomers, tautomer, isotopolog, or pharmaceutically acceptable salt thereof (e.g., a hydrochloride salt of Compound 1), is administered at a dose of from about 0.2 mg to about 0.6 mg once daily on days 1 to 10 of a 21-day cycle. In one embodiment, the compound is administered at a dose of about 0.2 mg once daily on days 1 to 10 of a 21-day cycle. In one embodiment, the compound is administered at a dose of about 0.3 mg once daily on days 1 to 10 of a 21-day cycle. In one embodiment, the compound is administered at a dose of about 0.4 mg once daily on days 1 to 10 of a 21-day cycle. In one embodiment, the compound is administered at a dose of about 0.5 mg once daily on days 1 to 10 of a 21-day cycle. In one embodiment, the compound is administered at a dose of about 0.6 mg once daily on days 1 to 10 of a 21-day cycle. In one embodiment, the administration period of the compound is followed by rest of the compound on days 11 to 21 of the 21-day cycle.

In one embodiment, provided herein is a method for treating aggressive B-cell lymphoma (a-BCL), comprising (i) administering Compound 1, or a pharmaceutically acceptable salt thereof (e.g., a hydrochloride salt of Compound 1), on days 1 to 7 of a 21-day cycle; (ii) administering rituximab, cyclophosphamide, doxorubicin, and vincristine on day 1 of the 21-day cycle; and (iii) administering prednisone or prednisolone on days 1 to 5 of the 21-day cycle. In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, provided herein is a method for treating aggressive B-cell lymphoma (a-BCL), comprising (i) administering Compound 1, or a pharmaceutically acceptable salt thereof (e.g., a hydrochloride salt of Compound 1), on days 1 to 10 of a 21-day cycle; (ii) administering rituximab, cyclophosphamide, doxorubicin, and vincristine on day 1 of the 21-day cycle; and (iii) administering prednisone or prednisolone on days 1 to 5 of the 21-day cycle. In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, provided herein is a method for treating aggressive B-cell lymphoma (a-BCL), comprising: (i) administering Compound 1, or a pharmaceutically acceptable salt thereof (e.g., a hydrochloride salt of Compound 1), orally at a dose of about 0.2 mg once daily on days 1 to 7 of a 21-day cycle; (ii) administering rituximab intravenously at a dose of about 375 mg/m² or subcutaneously at a dose of about 1400 mg on day 1 of the 21-day cycle; (iii) administering cyclophosphamide intravenously at a dose of about 750 mg/m² on day 1 of the 21-day cycle; (iv) administering doxorubicin intravenously at a dose of about 50 mg/m² on day 1 of the 21-day cycle; (v) administering vincristine intravenously at a dose of about 1.4 mg/m² on day 1 of the 21-day cycle; and (vi) administering prednisone or prednisolone orally at a dose of about 100 mg per day on days 1 to 5 of the 21-day cycle (day 1 intravenous administration of prednisone or prednisolone is acceptable). In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, provided herein is a method for treating aggressive B-cell lymphoma (a-BCL), comprising: (i) administering Compound 1, or a pharmaceutically acceptable salt thereof (e.g., a hydrochloride salt of Compound 1), orally at a dose of about 0.4 mg once daily on days 1 to 7 of a 21-day cycle; (ii) administering rituximab intravenously at a dose of about 375 mg/m² or subcutaneously at a dose of about 1400 mg on day 1 of the 21-day cycle; (iii) administering cyclophosphamide intravenously at a dose of about 750 mg/m² on day 1 of the 21-day cycle; (iv) administering doxorubicin intravenously at a dose of about 50 mg/m² on day 1 of the 21-day cycle; (v) administering vincristine intravenously at a dose of about 1.4 mg/m² on day 1 of the 21-day cycle; and (vi) administering prednisone or prednisolone orally at a dose of about 100 mg per day on days 1 to 5 of the 21-day cycle (day 1 intravenous administration of prednisone or prednisolone is acceptable). In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, provided herein is a method for treating aggressive B-cell lymphoma (a-BCL), comprising: (i) administering Compound 1, or a pharmaceutically acceptable salt thereof (e.g., a hydrochloride salt of Compound 1), orally at a dose of about 0.6 mg once daily on days 1 to 7 of a 21-day cycle; (ii) administering rituximab intravenously at a dose of about 375 mg/m² or subcutaneously at a dose of about 1400 mg on day 1 of the 21-day cycle; (iii) administering cyclophosphamide intravenously at a dose of about 750 mg/m² on day 1 of the 21-day cycle; (iv) administering doxorubicin intravenously at a dose of about 50 mg/m² on day 1 of the 21-day cycle; (v) administering vincristine intravenously at a dose of about 1.4 mg/m² on day 1 of the 21-day cycle; and (vi) administering prednisone or prednisolone orally at a dose of about 100 mg per day on days 1 to 5 of the 21-day cycle (day 1 intravenous administration of prednisone or prednisolone is acceptable). In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, provided herein is a method for treating aggressive B-cell lymphoma (a-BCL), comprising: (i) administering Compound 1, or a pharmaceutically acceptable salt thereof (e.g., a hydrochloride salt of Compound 1), orally at a dose of about 0.4 mg once daily on days 1 to 10 of a 21-day cycle; (ii) administering rituximab intravenously at a dose of about 375 mg/m² or subcutaneously at a dose of about 1400 mg on day 1 of the 21-day cycle; (iii) administering cyclophosphamide intravenously at a dose of about 750 mg/m² on day 1 of the 21-day cycle; (iv) administering doxorubicin intravenously at a dose of about 50 mg/m² on day 1 of the 21-day cycle; (v) administering vincristine intravenously at a dose of about 1.4 mg/m² on day 1 of the 21-day cycle; and (vi) administering prednisone or prednisolone orally at a dose of about 100 mg per day on days 1 to 5 of the 21-day cycle (day 1 intravenous administration of prednisone or prednisolone is acceptable). In one embodiment, the a-BCL is DLBCL (e.g., DLBCL, NOS (including GCB and ABC types). In one embodiment, the a-BCL is high-grade B-cell lymphoma (e.g., high-grade B-cell lymphoma with MYC and BCL2 and/or BCL6 rearrangements). In one embodiment, the a-BCL is PMBCL. In one embodiment, the a-BCL is primary cutaneous DLBCL-leg type. In one embodiment, the a-BCL is ALK+large B-cell lymphoma. In one embodiment, the a-BCL is EBV+DLBCL (e.g., EBV+DLBCL, NOS). In one embodiment, the a-BCL is grade 3b follicular lymphoma. In one embodiment, the a-BCL is previously untreated. In one embodiment, the a-BCL is newly diagnosed. In one embodiment, the a-BCL is poor risk a-BCL (e.g., with IPI score of 3 to 5).

In one embodiment, the method further comprises administering to the subject a growth factor. In one embodiment, the growth factor is administered for prophylactic purpose (e.g., to prevent a subject from developing neutropenia (e.g., grade ¾ neutropenia, prolonged severe neutropenia, febrile neutropenia)). In one embodiment, the growth factor is administered for therapeutic purpose (e.g., to treat or manage neutropenia (e.g., grade ¾ neutropenia, prolonged severe neutropenia, febrile neutropenia) in a subject that developed neutropenia). In one embodiment, the method further comprises administering to the subject granulocyte-colony stimulating factor (G-CSF) or pegylated granulocyte colony stimulating factor (peg-G-CSF).

In one embodiment, G-CSF is administered on days 5 to 13 of a 21-day cycle. In one embodiment, peg-G-CSF is administered on day 2 of a 21-day cycle.

In one embodiment, G-CSF is administered on days 5 to 13 of a 21-day cycle and a compound described herein, e.g., Compound 1, or an enantiomer, mixture of enantiomers, tautomer, isotopolog, or pharmaceutically acceptable salt thereof (e.g., a hydrochloride salt of Compound 1), is administered on days 1 to 7 of the 21-day cycle. In one embodiment, G-CSF is administered on days 5 to 13 of a 21-day cycle whereas a compound described herein, e.g., Compound 1, or an enantiomer, mixture of enantiomers, tautomer, isotopolog, or pharmaceutically acceptable salt thereof (e.g., a hydrochloride salt of Compound 1), is administered on days 1 to 10 of the 21-day cycle.

In one embodiment, peg-G-CSF is administered on day 2 of a 21-day cycle and a compound described herein, e.g., Compound 1, or an enantiomer, mixture of enantiomers, tautomer, isotopolog, or pharmaceutically acceptable salt thereof (e.g., a hydrochloride salt of Compound 1), is administered on days 1 to 7 of the 21-day cycle. In one embodiment, peg-G-CSF is administered on day 2 of a 21-day cycle whereas a compound described herein, e.g., Compound 1, or an enantiomer, mixture of enantiomers, tautomer, isotopolog, or pharmaceutically acceptable salt thereof (e.g., a hydrochloride salt of Compound 1), is administered on days 1 to 10 of the 21-day cycle.

In one embodiment, the method further comprises administering to the subject antithrombotic prophylaxis. In one embodiment, the method further comprises administering to the subject intrathecal (IT) prophylaxis for central nervous system (CNS) involvement.

In another embodiment, provided herein are methods for achieving a complete response, partial response, or stable disease, as determined by the Lugano response criteria in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in overall survival, progression-free survival, event-free survival, time to progression, or disease-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in overall survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in progression-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in event-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in time to progression in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In another embodiment, provided herein are methods for achieving an increase in disease-free survival in a patient, comprising administering an effective amount of a compound described herein, e.g., Compound 1, or an enantiomer, mixture of enantiomers, tautomer, isotopolog, or pharmaceutically acceptable salt thereof (e.g., a hydrochloride salt of Compound 1), in combination with a combination of rituximab, cyclophosphamide, doxorubicin, vincristine, and prednisone or an equivalent thereof, to patient having BCL. In one embodiment, the BCL is a-BCL.

### Pharmaceutical Compositions and Routes of Administration

The compound provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds in the pharmaceutical composition may be at a level that will exercise the desired effect; about 0.001 mg/kg of a subject's body weight to about 1 mg/kg of a subject's body weight in unit dosage for both oral and parenteral administration.

A compound provided herein can be administered orally. In one embodiment, when administered orally, a compound provided herein is administered with a meal and water. In another embodiment, the compound provided herein is dispersed in water or juice (e.g., apple juice or orange juice) and administered orally as a solution or a suspension. In one embodiment, a compound provided herein is administered when the subject is fed. In one embodiment, a compound provided herein is administered when the subject is fed with high-fat and/or high-calorie food. In one embodiment, a compound provided herein is administered when the subject is fed with FDA-standard high-fat high-calorie breakfast. In one embodiment, a compound provided herein is administered when the subject is fasted. In one embodiment, a compound provided herein is administered after the subject has an at least 8-hour overnight fast. In one embodiment, a compound provided herein is administered with or without food.

The compound provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound provided herein without an additional carrier, excipient or vehicle. In another embodiment, provided herein are compositions comprising an effective amount of a compound provided herein and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts. In general, all of the compositions are prepared according to known methods in pharmaceutical chemistry. Capsules can be prepared by mixing a compound provided herein with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound provided herein as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound provided herein can be delayed or prolonged by proper formulation. For example, a slowly soluble pellet of the compound provided herein can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound provided herein in oily or emulsified vehicles that allow it to disperse slowly in the serum.

Certain pharmaceutical compositions and formulations of Compound 1 are described in U.S. patent application Ser. No. 17/075,447, the entirety of which is incorporated herein by reference.

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old.

Depending on the state of the disease to be treated and the subject's condition, Compound 1, or an enantiomer, mixture of enantiomers, tautomer, isotopolog, or pharmaceutically acceptable salt thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Compound 1, or an enantiomer, mixture of enantiomers, tautomer, isotopolog, or pharmaceutically acceptable salt thereof, may be formulated, alone or together, in suitable dosage unit with pharmaceutically acceptable excipients, carriers, adjuvants and vehicles, appropriate for each route of administration.

In one embodiment, Compound 1, or an enantiomer, mixture of enantiomers, tautomer, isotopolog, or pharmaceutically acceptable salt thereof (e.g., a hydrochloride salt of Compound 1), is administered orally. In another embodiment, the compound of Compound 1, or an enantiomer, mixture of enantiomers, tautomer, isotopolog, or pharmaceutically acceptable salt thereof (e.g., a hydrochloride salt of Compound 1), is administered parenterally. In yet another embodiment, the compound of Compound 1, or an enantiomer, mixture of enantiomers, tautomer, isotopolog, or pharmaceutically acceptable salt thereof (e.g., a hydrochloride salt of Compound 1), is administered intravenously.

Compound 1, or an enantiomer, mixture of enantiomers, tautomer, isotopolog, or pharmaceutically acceptable salt thereof (e.g., a hydrochloride salt of Compound 1), can be delivered as a single dose such as, e.g., a single bolus injection, or oral capsules, tablets or pills; or over time, such as, e.g., continuous infusion over time or divided bolus doses over time. The compounds as described herein can be administered repeatedly if necessary, for example, until the patient experiences stable disease or regression, or until the patient experiences disease progression or unacceptable toxicity.

Compound 1, or an enantiomer, mixture of enantiomers, tautomer, isotopolog, or pharmaceutically acceptable salt thereof (e.g., a hydrochloride salt of Compound 1), can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered once or more than once each day, for example, for a period of time. The term “continuous” is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered daily for an uninterrupted period of at least 7 days to 52 weeks. The term “intermittent” or “intermittently” as used herein is intended to mean stopping and starting at either regular or irregular intervals. For example, intermittent administration of Compound 1, or an enantiomer, mixture of enantiomers, tautomer, isotopolog, or pharmaceutically acceptable salt thereof (e.g., a hydrochloride salt of Compound 1), is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound 1, or an enantiomer, mixture of enantiomers, tautomer, isotopolog, or pharmaceutically acceptable salt thereof (e.g., a hydrochloride salt of Compound 1), is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, Compound 1, or an enantiomer, mixture of enantiomers, tautomer, isotopolog, or pharmaceutically acceptable salt thereof (e.g., a hydrochloride salt of Compound 1), is administered once a day. In another embodiment, Compound 1, or an enantiomer, mixture of enantiomers, tautomer, isotopolog, or pharmaceutically acceptable salt thereof, is administered twice a day. In yet another embodiment, Compound 1, or an enantiomer, mixture of enantiomers, tautomer, isotopolog, or pharmaceutically acceptable salt thereof (e.g., a hydrochloride salt of Compound 1), is administered three times a day. In still another embodiment, Compound 1, or an enantiomer, mixture of enantiomers, tautomer, isotopolog, or pharmaceutically acceptable salt thereof (e.g., a hydrochloride salt of Compound 1), is administered four times a day.

In one embodiment, the methods provided herein include an administration of a therapeutically effective amount of Compound 1, or a pharmaceutically acceptable salt thereof (e.g., a hydrochloride salt of Compound 1) in one or more 21-day treatment cycles. In another embodiment, the methods provided herein include an administration of a therapeutically effective amount of Compound 1, or a pharmaceutically acceptable salt thereof (e.g., a hydrochloride salt of Compound 1) on days 1 to 10 of a 21-day cycle. In another embodiment, the methods provided herein include an administration of a therapeutically effective amount of Compound 1, or a pharmaceutically acceptable salt thereof (e.g., a hydrochloride salt of Compound 1) on days 1 to 14 of a 21-day cycle.

In one embodiment, Compound 1, or a pharmaceutically acceptable salt thereof (e.g., a hydrochloride salt of Compound 1) is administered once daily for 10 days followed by 11 days of rest. In one embodiment, Compound 1, or a pharmaceutically acceptable salt thereof (e.g., a hydrochloride salt of Compound 1) is administered once daily for 14 days followed by 7 days of rest.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 10 days followed by 11 days of rest, starting on day 1 of the first 21-day cycle; and (ii) administering a second therapeutic agent provided herein in cycles as described herein.

In one embodiment, the method provided herein comprises (i) administering a compound provided herein (e.g., Compound 1, or a pharmaceutically acceptable salt thereof, e.g., a hydrochloride salt of Compound 1) in cycles of once daily for 14 days followed by 7 days of rest, starting on day 1 of the first 21-day cycle; and (ii) administering a second therapeutic agent provided herein in cycles as described herein.

Any treatment cycle described herein can be repeated for at least 1, 2, 3, 4, 5, 6, 7, 8, or more cycles. In certain instances, the treatment cycle as described herein includes from 1 to about 24 cycles, from about 2 to about 16 cycles, or from about 2 to about 6 cycles. In certain instances a treatment cycle as described herein includes from 1 to about 4 cycles. In some embodiments, a therapeutically effective amount of Compound 1, or a pharmaceutically acceptable salt thereof (e.g., a hydrochloride salt of Compound 1), and/or a second therapeutic agent provided herein (e.g., a combination of rituximab, cyclophosphamide, doxorubicin, vincristine, and prednisone or an equivalent thereof) is administered for one or more 21-day cycles (e.g., six 21-day cycles). In certain instances, the cycling therapy is not limited to the number of cycles, and the therapy is continued until disease progression. Cycles can in certain instances include varying the duration of administration periods and/or rest periods described herein.

### EXAMPLES

The following Examples are presented by way of illustration, not limitation.

### Example 1: Phase 1b Clinical Study

A Phase 1b, open label, global, multicenter, dose determination, randomized dose expansion study to determine the maximum tolerated dose, assess the safety and tolerability, pharmacokinetics and preliminary efficacy of Compound 1 in combination with R—CHOP-21 is conducted for subjects with previously untreated, poor risk (IPI 3 to 5), aggressive B-cell lymphoma.

### Indication

Subjects with newly diagnosed aggressive B-cell lymphoma (a-BCL) are eligible for the study.

### Objectives

Primary objective for Part 1: To define the maximum tolerated dose (MTD) and/or the recommended Phase 2 dose (RP2D) of Compound 1 in combination with R—CHOP-21 in subjects with previously untreated, poor risk (IPI 3 to 5), a-BCL.

Primary objective for Part 2: To further evaluate the safety and tolerability associated with Compound 1 at the RP2D in combination with R—CHOP-21 in subjects with previously untreated, poor risk (IPI 3 to 5), a-BCL.

### Secondary Objectives for Part 1:

To determine the safety and tolerability of Compound 1 in combination with R—CHOP-21 in subjects with previously untreated, poor risk (IPI 3 to 5), a-BCL.
To characterize the pharmacokinetics (PK) of Compound 1 in combination with R—CHOP-21 in subjects with previously untreated, poor risk (IPI 3 to 5), a-BCL.
To evaluate the preliminary efficacy of Compound 1 in combination with R—CHOP-21 in subjects with previously untreated, poor risk (IPI 3 to 5), a-BCL.

Secondary objectives for Part 2:
To evaluate the preliminary efficacy of Compound 1 in combination with R—CHOP-21 in subjects with previously untreated, poor risk (IPI 3 to 5), a-BCL.
To characterize the pharmacokinetics (PK) of Compound 1 in combination with R—CHOP-21 in subjects with previously untreated, poor risk (IPI 3 to 5), a-BCL.

Exploratory objectives:
To correlate PK with safety profile, clinical activity, and pharmacodynamic (PD) biomarkers of Compound 1.
To evaluate the PK of Compound 1's R-enantiomer in subjects with previously untreated, poor risk (IPI 3 to 5), a-BCL.
To explore the relationship of dose, exposure, and response of Compound 1 to CRBN substrate degradation kinetics in tumors by fine needle aspirate.
To explore the relationship of dose, exposure, and response to CRBN substrate degradation kinetics in peripheral blood.
To explore the relationship of dose, exposure, and response of Compound 1 to peripheral immune cell populations by immunophenotyping.
To evaluate correlation of protein expression, gene expression or genomic abnormalities in tumor cells to Compound 1 response or relapse.
To evaluate the correlation of baseline and changes in immune cell composition of the tumor microenvironment to Compound 1 treatment and response or relapse.
To explore changes in circulating tumor DNA (ctDNA) levels and mutations and its correlation with radiological and metabolic response.
To assess the impact of severe acute respiratory syndrome coronavirus 2 (SARS-COV-2) on subjects receiving Compound 1 for the treatment of lymphoma and to support health authority requests.
To explore the correlation of biomarkers and efficacy endpoints.

### Study Design

This study is designed as a Phase 1b study consisting of 2 parts: a dose escalation (Part 1) of Compound 1 added to the standard R—CHOP-21 regimen for first-line treatment of a-BCL; and expansion at the RP2D (Part 2) of Compound 1 added to the standard R—CHOP-21 regimen for first-line treatment of a-BCL.

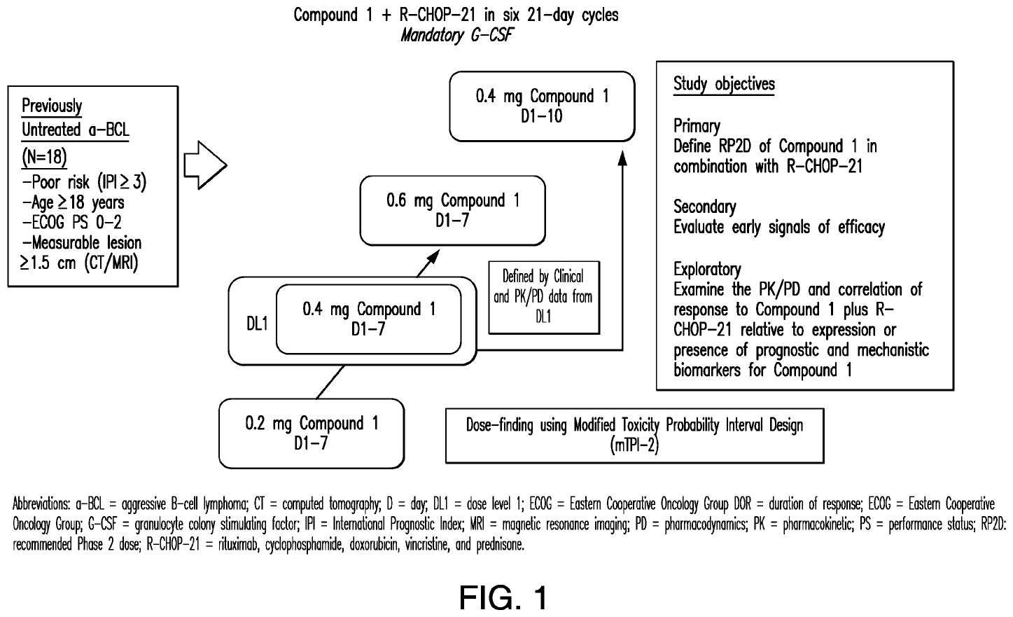

Part 1 (dose escalation) is to define the maximum tolerated dose (MTD) and/or the recommended Phase 2 dose (RP2D) and schedule of Compound 1 when given with standard doses of R—CHOP-21 for up to 6 cycles. Part 1 includes approximately 18 subjects treated with standard doses of R—CHOP-21 and escalating dose of Compound 1 (FIG. 1). Part 1 is conducted using a modified toxicity probability interval-2 (mTPI-2) design. Dose limiting toxicity (DLT) is assessed to determine the MTD/RP2D during the first 2 treatment cycles.

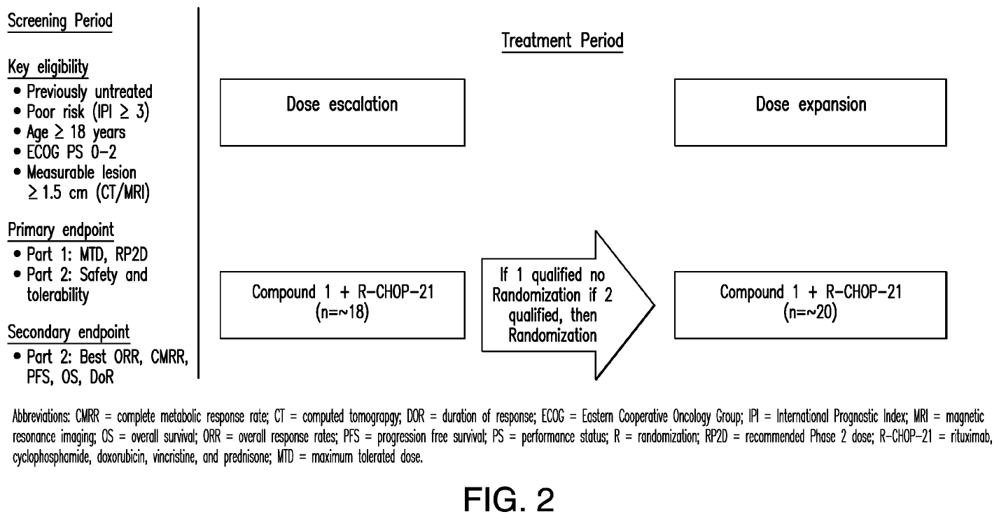

Part 2 (dose expansion): Once the RP2D is established for Compound 1 in Part 1, the corresponding Part 2 of the study is initiated. Approximately 20 subjects are enrolled and randomized into Compound 1 and R—CHOP-21 combination arm (FIG. 2).

The study is conducted in compliance with International Council for Harmonisation (ICH) Good Clinical Practices (GCPs).

### Study Population

Approximately 38 subjects are enrolled in the study (18 in Part 1 and 20 in Part 2).

Subjects must have an investigator-assessed diagnosis of a-BCL with IPI score of 3 to 5. Subjects (male or female) ≥18 years of age must have at least one measurable lesion according to Lugano classification of NHL (Cheson, 2014) and must have adequate bone marrow, liver, and renal function.

### Length of Study

The study consists of a screening, treatment and follow-up period for all subjects enrolled.

The expected duration of the entire study is 53 months, which includes an enrollment period of approximately 12 months for Part 1 and 12 months for Part 2 plus 5 months treatment plus 24 months follow-up.

The End of Trial is defined as either the date of the last visit of the last subject to complete the post-treatment follow-up, or the date of receipt of the last data point from the last subject that is required for primary, secondary and/or exploratory analysis, as prespecified in the protocol, whichever is the later date.

### Study Treatments

Compound (provided as a hydrochloride salt) is supplied as capsules for oral administration and labeled appropriately as investigational product (IP) for this study. Capsules of Compound 1 are administered orally (QD) on planned dosing days. Compound 1 must be administered in the morning with approximately 8 oz or 240 mL of water after an overnight fast lasting for at least 6 hours. Subjects must refrain from food or other medication intake for at least 2 hours after each morning dose.

Study treatment includes six 21-day cycles of R—CHOP-21 (intravenous (IV) or SC rituximab, doxorubicin, vincristine, and cyclophosphamide on Day 1; daily prednisone or prednisolone from Day 1 to Day 5), as shown in the Table 4 below. No other anthracycline may be substituted for doxorubicin. Liposomal doxorubicin is not permitted. In countries where prednisone is not available, it is acceptable to substitute the local equivalent corticosteroid. It is also acceptable to administer the corticosteroid intravenously rather than orally on Day 1 of the cycle for convenience.

TABLE 4
R-CHOP-21 Regimen

| | | | Dosing |
|---|---|---|---|
| | | | Day(s) |
| | | | (21-day |
| Drug | Dose | | cycle) |
| Rituximab IV or SC | 375 mg/m² | | 1 |
| | (IV) or 1400 mg (SC) | | |
| Cyclophosphamide IV | 750 | mg/m² | 1 |
| Doxorubicin IV | 50 | mg/m² | 1 |
| Vincristine IV | 1.4 mg/m² | | 1 |
| | (max of 2.0 mg total) | | |
| Prednisone or Prednisolone PO | 100 | mg | 1-5 |

(Day 1 IV administration is
acceptable)

In addition to R—CHOP-21, the following doses and schedules of orally administered Compound 1 are evaluated within the study:
DL1: 0.4 mg, Days 1 to 7
DL2a: 0.6 mg, Days 1 to 7
DL2b: 0.4 mg, Days 1 to 10
DL-1: 0.2 mg, Days 1 to 7

The decision to escalate to Dose Level 2a or 2b is based on clinical and PK/PD data from Dose Level 1.

Dose Level 2a and 2b deliver a similar total dose of Compound 1 per cycle, one with a higher dose delivered for a shorter interval, the other with a lower dose spreading out for a longer period. The first day of IP dosing of Compound 1 is considered Day 1 of a cycle.

Dosing interruptions and reductions are permitted throughout the study. Intrapatient dose escalation is not permitted in this study. Dose modification for management of toxicity is permitted.

Mandatory granulocyte colony stimulating factor (G-CSF) is given from Day 5 to Day 13 or pegylated granulocyte colony stimulating factor (peg-G-CSF) at Day 2 during each cycle. Antithrombotic prophylaxis is mandatory for all subjects in this study.

For subjects at poor risk, intrathecal (IT) prophylaxis for central nervous system (CNS) involvement is administered per institutional practice.

Study Endpoints

| Endpoint | Name | Description | Timeframe |
|---|---|---|---|
| Primary | Part 1: | Frequency of DLTs to define MTD | DLT evaluation period defined |
| | MTD and | and establish the RP2D of Compound | from first investigational |
| | RP2D | 1 in combination with R-CHOP-21 in | product dose through |
| | | subjects with previously untreated a- | completion of C2. |
| | | BCL. | |
| | Part 2: | Adverse events (AEs) evaluated using | From the first dose of any |
| | Safety and | NCI CTCAE criteria, v.5.0, including | investigational product until |
| | tolerability | treatment-emergent adverse events | 28 days after the last dose of IP. |
| | of | (TEAEs) and laboratory assessments. | |
| | Compound | | |
| | 1 at RP2D | | |
| Secondary | Efficacy | Best overall response rate (ORR) | After Cycle 3, at 6 to 8 weeks |
| | | Complete metabolic response rate | after EOT and then Months 9, |
| | | (CMRR) | 12, 18, and 24 after enrollment. |
| | | Time to response (TTR) | |
| | | Duration of response (DOR) | |
| | | Progression-free survival (PFS) | |
| | | Overall survival (OS) | |
| | | According to 2014 Lugano | |
| | | classification. | |
| | Safety and | Adverse events (AEs) evaluated using | From first dose to 28 days after |
| | tolerability | NCI CTCAE criteria, v.5.0, including | last dose of study treatment for |
| | | treatment-emergent adverse events | AEs occurring after 28 days |
| | | (TEAEs), laboratory assessments, and | from last dose of study |
| | | vital signs. | treatment that are suspected to |
| | | | be related to study treatment. |
| | PK | Cₘₐₓ, C_{trough}, AUC, tₘₐₓ, t½, CL/F, V/F | C1D7, C2D7. |
| | | for Compound 1. | |
| Exploratory | PK | To explore the relationship between | C1D7, C2D7. |
| | correlation | systemic exposure of Compound 1, | |
| | | PD biomarkers, and clinical activity. | |
| | PK | Cₘₐₓ, C_{trough}, AUC, tₘₐₓ, t½, CL/F, V/F | C1D7, C2D7. |
| | | for R-enantiomer of Compound 1. | |
| | PD | To explore relationship of dose, | Part 1 only - C1D1, C1D7, and |
| | | exposure, and response of Compound | C1D15. |
| | | 1 to CRBN substrate degradation | |
| | | kinetics in tumors by fine needle | |
| | | aspirate | |
| | PD | To explore relationship of dose, | Part 1 only - C1D1, C1D2, |
| | | exposure, and response to CRBN | C1D7, C1D15, and C2D1. |
| | | substrate degradation kinetics in | |
| | | peripheral blood | |
| | PD | To explore relationship of dose, | C1D1, C1D7, C1D15, C2D1, |
| | | exposure, and response of Compound | and C2D7 |
| | | 1 to peripheral immune cell | |
| | | populations by immunophenotyping | |
| | PD | Exploratory measurements of SARS- | Screening and EOT. |
| | | CoV-2 serology (anti-SARS-CoV-2 | |
| | | total of IgG), from serum samples | |
| | | collected at baseline and EOT. | |
| | Biomarkers | To evaluate correlation of protein | Part 1: Screening required, at |
| | | expression, gene expression or | progression desirable |
| | | genomic abnormalities in tumor cells | Part 2: Screening, C1D7, and at |
| | | to Compound 1 treatment and | progression. |
| | | response or relapse | |
| | Biomarkers | To evaluate the correlation of baseline | Part 1: Screening required, and |
| | | and changes in immune cell | at tumor progression desirable |
| | | composition of tumor | Part 2: Screening, C1D7, and at |
| | | microenvironment to Compound 1 | tumor progression. |
| | | treatment and response or relapse | |
| | Biomarkers | To explore changes in circulating | C1D1, C2D1, C4D1, Disease |
| | | tumor DNA (ctDNA) levels and | evaluation (CT, PET scan) |
| | | mutations and its correlation with | visits, 6 to 8 weeks after EOT, |
| | | radiological and metabolic | and at tumor progression. |
| | | response | |
| | Biomarkers | To explore the correlation of | 6 to 8 weeks after EOT; PFS at |
| | | biomarkers and efficacy endpoints | end of 2 years. |

a-BCL = aggressive B-cell lymphoma; AUC = area under the plasma concentration-time curve; C = Cycle; Cf = compare; CL/F = apparent oral clearance; Cₘₐₓ = maximum plasma concentration of drug; CT = computed tomography; C_{trough} = minimum or trough concentration; CTCAE = Common Terminology Criteria for Adverse Events; D = Day; DLT = dose-limiting toxicity; EOT = end of treatment; IgG = immunoglobulin G; IP = investigational product; MTD = maximum tolerated dose; NCI = National Cancer Institute; PET = positron emission tomography; PFS = progression-free survival; PK = pharmacokinetics; R-CHOP = rituximab, cyclophosphamide, doxorubicin, vincristine, and prednisone; RP2D = recommended Phase 2 dose; SARS-CoV-2 = severe acute respiratory syndrome coronavirus 2; t½ = terminal elimination half-life; tₘₐₓ = time to maximum plasma concentration of drug; V/F = apparent volume of distribution.

### Overview of Key Efficacy Assessments

Physical examination including Eastern Cooperative Oncology Group (ECOG) performance status.
Complete blood cell count.
Computed tomography (CT)-scan and/or fluorodeoxyglucose-positron emission tomography (FDG-PET)-CT scan.
Bone marrow evaluation: biopsy, aspiration.

### Overview of Key Safety Assessments

Complete physical examination including vital signs and venous thromboembolism (VTE) monitoring.
Clinical laboratory evaluations (hematology, serum chemistry).
Pregnancy testing/counseling.
Concomitant medications and procedures.
Adverse events (AEs).

### Statistical Methods

For each arm in the Part 1 portion of the study, a mTPI-2 design (Ji Y et al., Clin Trials, 7(6):653-63 (2010); Ji Y et al., J Clin Oncol, 31(14): 1785-91 (2013); Guo W et al., Contemp Clin Trials, 58:23-33 (2017)) is applied to guide the dose escalation. Approximately 18 subjects are enrolled in Compound 1 and R—CHOP-21 combination arm. The number of subjects depend on the number of dose levels being tested (based on the occurrence of DLT) and may exceed the approximations.

The target toxicity rate of DLT for the MTD is 0.25. Subjects are enrolled in groups of size ≥3 with maximum sample size of 9 for each dose level. The initial dose level of Compound 1 is 0.4 mg given for the first 7 days in combination with R—CHOP-21. The mTPI-2 algorithm with prior Beta (⅓, ⅓) and acceptable toxicity probability interval (0.2, 0.3) are applied to recommend the subsequent dose levels. The prior Beta(⅓, ⅓) is “neutral” in the sense that the maximum likelihood estimate of toxicity rate is approximately at the posterior median (Kerman J, http://arxiv.org/abs/1111.0433v1 (2011)).

The recommended dose is integrated with a clinical assessment of the toxicity profiles observed at the time of the analysis. To protect subject safety, the Safety Review Committee (SRC) oversees the dose escalation process and assess each dose level decision before it is assigned. The SRC may make recommendations to the Sponsor regarding the dose level assignment, independent of the dose recommended by the mTPI-2.

### Inclusion Criteria

Subjects must satisfy the following criteria to be enrolled in the study:
1. Subject is ≥18 years of age at the time of signing the informed consent form (ICF).
2. Subject must understand and voluntarily sign an ICF prior to any study-related assessments/procedures being conducted.
3. Subject is willing and able to adhere to the study visit schedule and other protocol requirements.
4. Subject is willing to undergo core needle or incisional/excisional biopsy unless sufficient tissue is available from diagnostic tumor/lymph node biopsy (from within 6 months prior to ICF signature) for translational research purposes.
5. Subject has histologically confirmed (per local evaluation) diagnosis of de novo, previously untreated, a-BCL according to 2016 WHO classification including:
   a. Diffuse large B-cell lymphoma (DLBCL), NOS (including germinal center B-cell [GCB] and activated B-cell [ABC] types);
   b. High-grade B-cell lymphoma, with MYC and BCL2 and/or BCL6 rearrangements;
   c. Primary mediastinal (thymic) large B-cell lymphoma (PMBCL);
   d. Primary cutaneous DLBCL-leg type;
   e. Anaplastic lymphoma kinase positive (ALK+) large B-cell lymphoma;
   f. Epstein Barr virus positive (EBV+) DLBCL, NOS;
   g. Grade 3b follicular lymphoma (FL).
6. Subject is considered an appropriate candidate (per investigator assessment) for induction therapy with 6 cycles of R—CHOP-21 immunochemotherapy.
7. Subject has poor-risk disease defined as International Prognostic Index (IPI) score ≥3 (high-intermediate or high-risk).
8. Subjects must have measurable disease defined by at least one FDG-avid lesion for FDGavid subtype and one bi-dimensionally measurable (>1.5 cm in longest diameter) disease by computed tomography (CT) or magnetic resonance imaging (MRI), as defined by the Lugano classification (Cheson, 2014).
9. Subject has an Eastern Cooperative Oncology Group (ECOG) performance status of 0, 1, or 2.
10. Subjects must have the following laboratory values:
   a. Absolute neutrophil count (ANC) ≥1.5×109/L or ≥1.0×109/L in case of documented bone marrow involvement (>50% or tumor cells), without growth factor support for 7 days (14 days if peg-G-CSF);
   b. Hemoglobin (Hb) ≥8 g/dL;
   c. Platelets (PLT) ≥75×109/L or ≥50×109/L in case of documented bone marrow involvement (>50% or tumor cells), without transfusion for 7 days;
   d. Aspartate aminotransferase/serum glutamic oxaloacetic transaminase (AST/SGOT) and alanine aminotransferase/serum glutamate pyruvic transaminase (ALT/SGPT)≤2.5× upper limit of normal (ULN). In the case of documented liver involvement by lymphoma, ALT/SGPT and AST/SGOT must be ≤5.0×ULN.
   e. Serum total bilirubin ≤2.0 mg/dL (34 μmol/L) except in cases of Gilbert syndrome, then ≤5.0 mg/dl (86 μmol/L);
   f. Estimated serum creatinine clearance of ≥50 mL/min using the modification of diet in renal disease (MDRD) formula.
11. All subjects must:
   a. Have an understanding that the study drug could have a potential teratogenic risk.
   b. Agree to refrain from donating blood while on study treatment, during dose interruptions and for at least 28 days following the last dose of study treatment.
   c. Agree not to share study medication with another person.
   d. Agree to follow all requirements defined in the Pregnancy Prevention Program for Compound 1 Pregnancy Prevention Plan for Subjects in Clinical trials.
12. Females must agree to abstain from breastfeeding during study participation and for at least 28 days after Compound 1 discontinuation and according to the approved rituximab product/prescribing information.
13. Females of childbearing potential (FCBP\hat{ })* must:
   a. Have two negative pregnancy tests as verified by the investigator prior to starting study therapy. She must agree to ongoing pregnancy testing during the course of the study, and after end of study therapy. This applies even if the subject practices true abstinence* from heterosexual contact.
   b. Either commit to true abstinence* from heterosexual contact (which must be reviewed on a monthly basis and source documented) or agree to use, and be able to comply with two forms of contraception: one highly effective, and one additional effective (barrier) measure of contraception without interruption 28 days prior to starting IP, during the study treatment (including dose interruptions), and for at least 28 days after the last dose of Compound 1 and for 12 months after the last dose of rituximab, whichever is longer.
14. Male subjects must:
   a. Practice true abstinence* (which must be reviewed on a monthly basis and source documented) or agree to use a condom during sexual contact with a pregnant female or a female of childbearing potential while participating in the study, during dose interruptions and for at least 90 days after the last dose of Compound 1, or rituximab, whichever is longer, even if he has undergone a successful vasectomy.
   b. Must agree to refrain from donating sperm while on study treatment, during dose interruptions, and for at least 90 days following last dose of Compound 1 or rituximab, whichever is longer.
      \hat{ }A FCBP is a female who: 1) has achieved menarche at some point, 2) has not undergone a hysterectomy or bilateral oophorectomy, or 3) has not been naturally postmenopausal (amenorrhea following cancer therapy does not rule out childbearing potential) for at least 24 consecutive months (i.e., has had menses at any time in the preceding 24 consecutive months). *True abstinence is acceptable when this is in line with the preferred and usual lifestyle of the subject. Periodic abstinence (e.g., calendar, ovulation, symptothermal, post-ovulation methods) and withdrawal are not acceptable methods of contraception.

### Exclusion Criteria

The presence of any of the following excludes a subject from enrollment:
1. Subject has any significant medical condition, active infection (including SARS-COV-2 suspected or confirmed), laboratory abnormality, or psychiatric illness that would prevent the subject from participating in the study.
2. Subject has any condition including the presence of laboratory abnormalities, which places the subject at unacceptable risk if he/she were to participate in the study.
3. Subject has any condition that confounds the ability to interpret data from the study.
4. Subject has any other subtype of lymphoma.
5. Subject has documented or suspected CNS involvement by lymphoma.
6. Subject has persistent diarrhea or malabsorption ≥Grade 2 (NCI CTCAE v5.0), despite medical management.
7. Subject has peripheral neuropathy ≥Grade 2 (NCI CTCAE v5.0).
8. Subject is on chronic systemic immunosuppressive therapy or corticosteroids (e.g., prednisone or equivalent not to exceed 10 mg per day within the last 14 days); stable use of inhaled or topical corticosteroids is allowed.
9. Subject has impaired cardiac function or clinically significant cardiac disease, including any of the following:
   a. Left ventricular ejection fraction (LVEF)<45% as determined by multigated
   b. acquisition scan (MUGA) or echocardiogram (ECHO);
   c. Heart failure (New York Heart Association Class III or IV);
   d. Clinically significant abnormal electrocardiogram (ECG) finding at screening;
   e. Unstable angina or myocardial infarction ≤6 months prior to starting;
   f. Persistent or uncontrolled ventricular arrhythmias or atrial fibrillation or cardiac conduction abnormalities not mitigated by a pacemaker.
10. Subject had major surgery ≤2 weeks prior to starting Compound 1; subject must have recovered from any clinically significant effects of recent surgery.
11. Subject has any condition causing inability to swallow tablets.
12. Subject has known seropositivity for or active viral infection with human immunodeficiency virus (HIV);
13. Subject has known chronic active hepatitis B (hepatitis B surface antigen [HBsAg] positive and/or hepatitis B core antibody [anti-HBc] positive with viral DNA positive) or C (positive serology requiring treatment and/or with evidence of liver damage) infection;
14. Subject has history of other malignancy, unless being free of the disease for ≥3 years; exceptions to the ≥3-year time limit include history of the following:
   a. Localized nonmelanoma skin cancer;
   b. Carcinoma in situ of the cervix;
   c. Carcinoma in situ of the breast;
   d. Incidental histologic finding of prostate cancer (T1a or T1b as per Tumor Node Metastasis [TNM] staging system) or prostate cancer that has been treated with curative intent.
15. Subject has current treatment with strong CYP3A4/5 modulators.
16. Subject has hypersensitivity to the active substance or to murine proteins, or to any of the other excipients of rituximab.
17. Subject has known hypersensitivity to any component of CHOP regimen.
18. Subject has known allergy to thalidomide, pomalidomide, or lenalidomide.

A number of references have been cited, the disclosures of which are incorporated herein by reference in their entirety.

The embodiments described above are intended to be merely exemplary, and those skilled in the art will recognize, or are able to ascertain using no more than routine experimentation, numerous equivalents of specific compounds, materials, and procedures. All such equivalents are considered to be within the scope of the invention and are encompassed by the appended claims.

What is claimed is:

1. A method of treating B-cell lymphoma (BCL), comprising administering to a subject in need thereof a therapeutically effective amount of a compound of Formula (I): 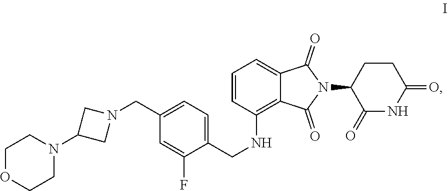 [SMILES: O=C1CC[C@H](N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, in combination with a second therapeutic agent, wherein the second therapeutic agent is a combination of rituximab, cyclophosphamide, doxorubicin, vincristine, and prednisone or an equivalent thereof.

2. The method of claim 1, wherein the BCL is aggressive B-cell lymphoma (a-BCL).

3. The method of claim 2, wherein the a-BCL is diffuse large B-cell lymphoma (DLBCL).

4. The method of claim 3, wherein the DLBCL is DLBCL not otherwise specified (NOS).

5. The method of claim 3 or 4, wherein the DLBCL is germinal center B-cell (GCB) type or activated B-cell (ABC) type.

6. The method of claim 2, wherein the a-BCL is high-grade B-cell lymphoma.

7. The method of claim 6, wherein the high-grade B-cell lymphoma has MYC and BCL2 and/or BCL6 rearrangements.

8. The method of claim 2, wherein the a-BCL is primary mediastinal (thymic) large B-cell lymphoma (PMBCL).

9. The method of claim 2, wherein the a-BCL is primary cutaneous DLBCL-leg type.

10. The method of claim 2, wherein the a-BCL is anaplastic lymphoma kinase positive (ALK+) large B-cell lymphoma.

11. The method of claim 2, wherein the a-BCL is Epstein Barr virus positive (EBV+) DLBCL.

12. The method of claim 11, wherein the EBV+DLBCL is EBV+DLBCL not otherwise specified (NOS).

13. The method of claim 2, wherein the a-BCL is Grade 3b follicular lymphoma (FL).

14. The method of claim 2, wherein the a-BCL is T-cell/histiocyte-rich large B-cell lymphoma (THRLBCL), intravascular large B-cell lymphoma, plasmablastic lymphoma, or primary effusion lymphoma (PEL).

15. The method of any one of claims 1 to 14, wherein the BCL has International Prognostic Index (IPI) score of 3 to 5.

16. The method of any one of claims 1 to 15, wherein the BCL is previously untreated.

17. The method of any one of claims 1 to 15, wherein the BCL is newly diagnosed.

18. The method of any one of claims 1 to 17, wherein the second therapeutic agent is a combination of rituximab, cyclophosphamide, doxorubicin, vincristine, and prednisone.

19. The method of any one of claims 1 to 17, wherein the second therapeutic agent is a combination of rituximab, cyclophosphamide, doxorubicin, vincristine, and prednisolone.

20. The method of any one of claims 1 to 19, wherein rituximab, cyclophosphamide, doxorubicin, and vincristine are administered on day 1 of a 21-day cycle, and prednisone or an equivalent thereof is administered on days 1 to 5 of the 21-day cycle.

21. The method of any one of claims 1 to 20, wherein rituximab is administered intravenously or subcutaneously; cyclophosphamide, doxorubicin, and vincristine are administered intravenously; and prednisone or an equivalent thereof is administered orally.

22. The method of any one of claims 1 to 21, wherein rituximab is administered intravenously at a dose of about 375 mg/m², or subcutaneously at a dose of about 1400 mg, on day 1 of a 21-day cycle; cyclophosphamide is administered intravenously at a dose of about 750 mg/m² on day 1 of the 21-day cycle; doxorubicin is administered intravenously at a dose of about 50 mg/m² on day 1 of the 21-day cycle; vincristine is administered intravenously at a dose of about 1.4 mg/m² on day 1 of the 21-day cycle; and prednisone or an equivalent thereof is administered orally at a dose of about 100 mg on days 1 to 5 of the 21-day cycle.

23. The method of any one of claims 1 to 22, wherein a compound of Formula (I), or a pharmaceutically acceptable salt thereof, is administered.

24. The method of claim 23, wherein a hydrochloride salt of a compound of Formula (I) is administered.

25. The method of any one of claims 1 to 24, wherein the compound is administered orally.

26. The method of any one of claims 1 to 25, wherein the compound is administered at a dose of from about 0.2 mg to about 0.6 mg once daily (QD).

27. The method of claim 26, wherein the compound is administered at a dose of about 0.2 mg, about 0.4 mg, or about 0.6 mg once daily (QD).

28. The method of any one of claims 1 to 27, wherein the compound is administered
(a) at a dose of about 0.2 mg QD on days 1 to 7 of a 21-day cycle;
(b) at a dose of about 0.4 mg QD on days 1 to 7 of a 21-day cycle;
(c) at a dose of about 0.4 mg QD on days 1 to 10 of a 21-day cycle; or
(d) at a dose of about 0.6 mg QD on days 1 to 7 of a 21-day cycle;

29. The method of claim 1, for treating a-BCL, comprising (i) administering the compound on days 1 to 7 of a 21-day cycle; (ii) administering rituximab, cyclophosphamide, doxorubicin, and vincristine on day 1 of the 21-day cycle; and (iii) administering prednisone or prednisolone on days 1 to 5 of the 21-day cycle.

30. The method of claim 1, for treating a-BCL, comprising (i) administering the compound on days 1 to 10 of a 21-day cycle; (ii) administering rituximab, cyclophosphamide, doxorubicin, and vincristine on day 1 of the 21-day cycle; and (iii) administering prednisone or prednisolone on days 1 to 5 of the 21-day cycle.

31. The method of any one of claims 1 to 30, wherein the method further comprises administering to the subject granulocyte-colony stimulating factor (G-CSF) or pegylated granulocyte colony stimulating factor (peg-G-CSF).

32. The method of claim 31, wherein G-CSF is administered on days 5 to 13 of a 21-day cycle whereas the compound is administered on days 1 to 7 of the 21-day cycle; or G-CSF is administered on days 5 to 13 of a 21-day cycle whereas the compound is administered on days 1 to 10 of the 21-day cycle.

33. The method of claim 31, wherein peg-G-CSF is administered on day 2 of a 21-day cycle whereas the compound is administered on days 1 to 7 of the 21-day cycle; or peg-G-CSF is administered on day 2 of a 21-day cycle whereas the compound is administered on days 1 to 10 of the 21-day cycle.

34. The method of any one of claims 1 to 33, wherein the compound, rituximab, cyclophosphamide, doxorubicin, vincristine, and prednisone or an equivalent thereof are administered in one or more 21-day cycles.

35. The method of claim 34, wherein the compound, rituximab, cyclophosphamide, doxorubicin, vincristine, and prednisone or an equivalent thereof are administered in six 21-day cycles.
